(19) United States
(12) Patent Application Publication
(10) Pub. No.: US 2011/0319495 A1
(43) Pub. Date: Dec. 29, 2011

(54) Pharmaceutical Composition for Hepatitis Treatment and Applications

(76) Inventor: Sheng-Yang Wang, New Taipei City (TW)

(21) Appl. No.: 12/826,257

(22) Filed: Jun. 29, 2010

(51) Int. Cl. A61K 31/122; A61P 29/00

(52) U.S. Cl. 514/690

(57) ABSTRACT

A pharmaceutical composition for hepatitis treatment, which comprises lucidone as an active ingredient; and a pharmaceutical acceptable carrier. As lucidone is a natural cyclopentenedione, the pharmaceutical composition of present invention has lesser side effects. The present invention is also related to the application of the pharmaceutical composition.

### BACKGROUND OF INVENTION

1. Field of the Invention

The present invention relates generally to a pharmaceutical composition for hepatitis treatment, and more particularly to an innovative one which is used to cure alcoholic hepatitis.

2. Description of Related Art

Hepar, one of most important human organs, is available with many important physiological functions, such as drug metabolism, amino acid metabolism, lipid metabolism and Glycolysis. Despite of the fact that acute and chronic Liver diseases raise common concern, existing treatment methods for liver diseases have to be further improved for better effect. Take alcoholic liver disease (ALD), for example, some researches prove that heme oxygenase, tumor necrosis factor (TNF) and reactive oxygen species (ROS) are closely related to alcoholic liver disease, but there lack of efficient treatment methods for alcoholic liver diseases.

Recently, great importance is attached to the risks caused by the side effects of synthetic chemical drugs, and alternative drugs based on natural compounds have drawn the attention of the academia and medical field. Lucidone is a natural cyclopentenedione extracted from the fruit of Lauraceae, which is believed to have great potential of antioxidation; however, no research has demonstrated the effect of lucidone for treatment of alcoholic liver diseases.

### CONTENT OF INVENTION

The primary objective of the present invention is to provide a pharmaceutical composition for hepatitis treatment, and more particularly to one for efficient treatment of alcoholic hepatitis.

Another objective of the present invention is to provide a pharmaceutical composition for hepatitis treatment, which takes natural compound as the active ingredient, so as to reduce the risks of side effect.

For the aforementioned purposes, the present invention provides a pharmaceutical composition for hepatitis treatment, which comprises effective quantity of lucidone as active ingredient; and a pharmaceutical acceptable carrier.

Preferably, said hepatitis refers to alcoholic hepatitis.

Preferably, said pharmaceutical composition comprises 1˜2 wt % lucidone, and 98˜99 wt % pharmaceutical acceptable carrier.

Preferably, said carrier refers to starch, lactose, cane sugar, MCC or CMC.

Preferably, said pharmaceutical composition further comprises pharmaceutical acceptable additive, which refers to amino acid, vitamin or mineral substance.

Preferably, said pharmaceutical composition refers to troche, capsule, skin patch, suspending agent, powder, nasal inhaler, spray or injection.

The present invention also provides a method of reducing the oxidation stress caused by alcohol, which enables contact of the sample with said pharmaceutical composition.

Preferably, said sample refers to mammals.

Preferably, said mammals refer to human being.

To sum up, the present invention is related to a pharmaceutical composition, which comprises lucidone as an active ingredient for hepatitis and alcoholic hepatitis treatment. As lucidone is a natural cyclopentenedione, the pharmaceutical composition of present invention can reduce greatly the side effects.

### SUMMARY OF THE INVENTION

The existing researches show that alcohol will cause inflammation of the liver along with increasing oxidation stress and inflammatory responses, such as: reduction of glutathione content, increase of lipid peroxidation as well as tumor necrosis factor-α (TNF-α), nitric oxide, reactive oxygen species and prostaglandin-E₂ in the blood plasma. In addition, the expression of heme oxygenase and the activation of transcription factor NF-E2 related factor-2 in the cells are considered as an index of growing oxidation stress; aspartate aminotransferase (AST) and Alanine aminotransferase (ALT) are two ferments in the liver cell, which are discharged to the serum only when the liver cells are damaged and broken. Thus, the growing content of AST and ALT in serum is regarded as an important indicator of liver malfunction (liver inflammation).

“Treatment” referred herein means treatment of the hepatitis objects with the pharmaceutical composition of the present invention; more specifically, it means treatment of the objects suffered from alcoholic hepatitis, or the individuals with hepatitis via the help of the pharmaceutical composition of the present invention. It is aimed at curing, mitigating, reducing, preventing and improving the symptom; in specific terms, “hepatitis treatment” referred herein is intended for reducing the content of or preventing the increase of AST and ALT in the serum. The “active ingredient” referred herein means the ingredient in the pharmaceutical composition that can generate actually aforementioned treatment effect. The “effective quantity” referred herein means the dose that's available with the treatment effect by using independently or by combining the pharmaceutical composition of the present invention.

The “pharmaceutical acceptable carrier” of the present invention is selected from commonly-used carriers without affecting the effect of active ingredient in the pharmaceutical composition of the present invention, including but not limited to: starch, lactose, cane sugar, MCC or CMC. The pharmaceutical composition of the present invention can be added with pharmaceutical acceptable additive to supplement the nutrition for the individuals of the present invention, including but not limited to: amino acid, vitamin or mineral substance; of which vitamin includes but not limited to: vitamin B group, vitamin C, vitamin E or their combinations. The pharmaceutical composition of the present invention refers to troche, capsule, skin patch, suspending agent, powder, nasal inhaler, spray or injection. In principle, the pharmaceutical composition of the present invention can be fed in any typical way, or preferably by oral administration. The dose of the pharmaceutical composition of the present invention will depend on the age, course of treatment and/or degree of symptom of the patients.

### BRIEF DESCRIPTION OF THE DRAWINGS

FIG. 1A shows the test result of lucidone's cell toxicity in HepG2 cell model.

FIG. 1B shows the test result of alcohol's cell toxicity in HepG2 cell model.

FIG. 2A shows the content of AST in HepG2 cell model.

FIG. 2B shows the content of ALT in HepG2 cell model.

FIG. 2C shows the content of TNF-α in HepG2 cell model.

FIG. 3A shows the content of reactive oxygen species in HepG2 cell model.

FIG. 3B shows the content of nitric oxide in HepG2 cell model.

FIG. 4A shows the content of GSH in HepG2 cell model.

FIG. 4B shows the lipid peroxidation degree in HepG2 cell model.

FIG. 5A shows the protein expression of HO-1 in HepG2 cell model.

FIG. 5B shows the mRNA expression of HO-1 in HepG2 cell model.

FIG. 6A shows the degree of Nrf-2 translocation in HepG2 cell model.

FIG. 6B shows the bonding activity of Nrf-2 and ARE in HepG2 cell model.

### PREFERRED EMBODIMENT 1

### Experimental Materials and Methods

### [Chemical Reagent and Anti-Body]

The lucidone of the present invention is purified by typical methods, with its purity up to 99% after identification by high-performance liquid chromatography (HPLC) and 1H-NMR. Alcohol, curcumin (purity 99%) and Griess reagent are purchased from Sigma-Aldrich (St. Louis, Mo., USA). Mouse anti-HO-1 monoclonal anti-body and rabbit anti-Nrf-2 multiple-clonal anti-body are purchased from Abcam (Cambridge, Mass., USA). Mouse-anti actin monoclonal anti-body is purchased from Sigma-Aldrich (St. Louis, Mo., USA).

### [Experimental Mouse]

HepG2 (human hepatocarcinoma cell, (purchased from American Type Culture Collection (ATCC, Manassas, Va., USA)) is only cultivated within MEM (minimum essential medium) in normal state, and added with 10% FBS, 4.5 g/L glucose, 4 mM glutamine, penicillin (100 units/mL) and streptomycin (100 μg/mL) to supplement the nutrition and necessary antibiotics, then placed in the environment of 37° C. and 5% CO₂. According to the experimental plan, HepG2 cells cultivated normally will be cultivated together with different concentrations of lucidone (1, 5 or 10 μg/mL) and curcumin (10 μg/mL), and if required by the experimental design, added with 100 mM alcohol for cultivation of 12-24 h.

### [Experimental Mouse]

The experimental mouse is selected from 4-week male mouse of ICR family (25±5 g) in Charles River (Taipei, Taiwan). Prior to the experiment, the experimental mouse will be bred for at least 1 week in constant temperature lab conditions wherein unlimited feedstock and water are supplied. All animals shall be governed by the Guide for the Care and Use of Laboratory Animals and relevant animal protection acts of Taiwan, and approved by local ethics commissions. 80% alcohol or lucidone is applied to aforementioned experimental mouse (1 mL/Kg) by following methods: alcohol is applied orally, or by intra-peritoneal injection, or alcohol and lucidone are applied orally, or by intra-peritoneal injection.

### [MTT Experiment]

MTT (3-[4,5-dimethyl-thiazol-2-yl]-2,5-diphenyl tetrazolium bromide) referred herein is purchased from Sigma-Aldrich (St. Louis, Mo., USA). MTT experiment indicates a common method of testing biologically the cell viability or multiplication. According to the principle, the succinate dehydrogenase in the mitochondria of living cells is used to metabolize tetrazolium of MTT into bluish violet product; next, DMSO is added to dissolve the bluish violet metabolic product stacked in the cells, then the quantity of bluish violet metabolic product can be measured using spectrometer, so as to estimate indirectly the number of living cells. By using the common procedures, MTT experiment allows to cultivate aforementioned HepG2 24 h and then remove the culture solution. Next, fresh MEM culture solution mixed with MTT (10 μl MTT (10 μg/mL) mixed with 90 μl fresh MEM culture solution) is added to the culture tray, and then aforementioned HepG2 cells are bred 1 h in 37° C. Finally, a spectrometer (ELISA microplate reader, μQuant, Bio-Tek instruments, Inc., Winooski, Vt., USA) is used to measure the quantity of bluish violet metabolic product at wavelength 570 nm to estimate indirectly the cell viability.

### [Test of ALT and AST Concentration]

A currently available test kit (Randox Laboratories, Antrim, UK) is applied to test the concentration of culture solution after cultivation of HepG2 cells as well as the concentration of ALT and AST in mouse serum. In brief, the experiment is designed to obtain the culture solution after cultivation of HepG2 cells and the serum of experimental mouse by the recommended test procedures. Then, a spectrometer (ELISA microplate reader, μQuant, Bio-Tek instruments, Inc., Winooski, Vt., USA) is used to judge the absorbance at wavelength 340 nm, thereby estimating the concentration of ALT and AST, which is represented by katal (U/L).

### [Test of Lipid Peroxidation Degree]

A currently available lipid peroxidation test kit (Oxford Biomedical Research, Oxford, Mich., USA) is used to measure the degree of lipid peroxidation. In brief, the cells are lysed by 200 μl cell lysis buffer. Then, 140 μl cell lysate is mixed with 455 μl N-methyl-2-phenylindole dissolved in acetonitrile and 105 μl acid (HCL, 37%), and placed in 45° C. for 60 m. malondialdehyde (MDA) is a main product of polyunsaturated fatty acid lysed by peroxidation. In this experiment, N-methyl-2-phenylindole is used to generate stable chromophore through reaction with MDA, and the formation of MDA is measured under maximum absorbance wavelength 535 nm as an index of measuring lipid peroxidation. Similarly, the degree of lipid peroxidation in the serum of experimental mouse can be measured.

### [Test of Glutathione Content]

A currently available GSH-96-well plate colorimetric GSH assay kit, Oxis International, Foster City, Calif., USA) is used to measure the content of glutathione (GSH). In brief, 40 μl chromogenic reagent dissolved in acid is added into 40 μl non-protein hepatocyte lysate, and added with 40 μl NaOH (30%), 40 μl color developer and buffer solution to form a mixed solution. Next, the mixed solution is shaken gently, and placed in room temperature for 10 m. Finally, a spectrometer (ELISA microplate reader, μQuant, Bio-Tek instruments, Inc., Winooski, Vt., USA) is used to detect the absorbance at wavelength 412 nm, and compared with a GSH absorbance curve of known concentration to judge the content of GSH. Similarly, the content of glutathione in the experimental mouseserum can be measured.

### [Griess Nitrite Test and Measurement of Tumor Necrosis Factor-α]

According to the principle of Griess reaction, nitrite accumulated from the supernatant liquid cultivated by cells and mouse serum is measured to obtain indirectly the generation of nitric oxide. The supernatant liquid cultivated by cells and tumor necrosis factor-α in the serum are measured using the currently available human TNF-αELISA kit. In brief, aforementioned HepG2 cells of 1×10⁷ cells/well are separately cultivated in 96-well plate, and then added with lucidone and alcohol to cultivate them for 24 h. Next, the culture solution is diluted by the work reagent of aforementioned kit by a percentage of 1:2. Then, every 100 μl diluted culture solution is subject to ELISA reaction as per the operating instruction of aforementioned kit, and then a spectrometer is used to detect the absorbance at wavelength 450 nm so as to judge the content of tumor necrosis factor-α; in the mouse experiment, the mouse serum is diluted properly and then the content of tumor necrosis factor-α is tested as per the operating instruction of aforementioned kit.

### [Test of Accumulation of Reactive Oxygen Species in Cells]

A currently available DCF-DA (dihydrodichloro-fluorescein diacetate; Sigma, USA) is used to monitor the accumulation of reactive oxygen species (ROS) in cells. In brief, the cells (1×10⁷) are wetted by phosphate buffer solution, and then added into 10 μm DCF-DA for 4 h cultivation at 37° C., and then fluorescence spectrophotometer is used to measure the fluorescence (488 nm/530 nm).

### [Preparation of Cytosolic Extracts and Nuclear Extracts]

The cytosolic extracts and nuclear extracts are prepared as per the known test procedures using the currently available test kit (protocol #78833 Nuclear and Cytoplasmic Extraction Reagent kit). And, the common protein assay reagent (Bio-Rad) is used to measure the concentration of protein.

### [Western Blotting]

Western blotting is implemented as per the known test procedures in the relevant field, of which said anti-body and enhanced chemiluminescene regents (ECL, Pierce) are used for immunoblotting, and then an imaging device (Viogene Biotek, Sunnyvale, Calif., USA) is used for observing the results.

### [Electrophoretic Mobility Shift Assay]

Electrophoretic mobility shift assay (EMSA) is implemented with a reference to know test procedures. In brief, the oligonucleotide probe: Nrf-2 positive strand 5′-CAG CAG GAC ATG GAT TTG ATT GAC-3′, and reverse strand 5′-AGA AAA GGC TCC ATC CTC CCG AAC-3′ are synthesized by TRI Biotech (Taipei, Taiwan), then heated 5 m in TE buffer solution at 94° C., and cooled down slowly 3 h for bonding two strands. Nucleus extract (20 μg/ml) and 20 ng Nrf-2 oligonucleotides (added with 5 μl binding buffer) are placed at room temperature for 30 m, and 6% native polyacrylamide gel is used for separating DNA/protein compound, next Light-Shift Chemiluminescent EMSA Kit (Pierce Biotechnology Inc., Rockford, USA) is used for imaging of the compound, and VL Chemi-Smart 3000 (Viogene Biotek) is employed to capture the strength image of cold light.

### [RNA Extraction and Real-Time PCR]

Trizol reagent (Invitrogen Life Technologies, Carlsbad, Calif., USA) is used to extract total RNA of cells. One step RT-PCR is used for reverse transcription of mRNA of HO-1 and β-actin into cDNA, then real time-PCR (Applied Biosystems) is applied to real-time expression of HO-1 and β-actin.

Said real-time PCR is used to detect PCR products by combining DNA and SYBR Green. The thermal cycle is: 3 m at 96° C.; 1 m at 96° C. in 40 cycles, 30 s at 50° C. and 90 s at 72° C. The sequence of primer is: HO-1 positive strand 5′-TGC GGT GCA GCT CTT CTG-3′; HO-1 reverse strand 5′-GCA ACC CGA CAG CAT GC-3′; β-actin positive strand 5′-ACC CAC ACT GTG CCC ATC TA-3′; β-actin reverse strand 5′-CGG AAC CGC TCA TTG CC-3′. β-actin, a housekeeping gene, is a stable gene of overexpression in cells, and used to standardize the expression of HO-1 in the experiments.

### PREFERRED EMBODIMENT 2

### Discussion of the Effects of Lucidone Against Alcoholic Oxidation Stress and Hepatitis with HepG2 Cell Model

In this preferred embodiment, HepG2 is taken as a model system to examine the effects of lucidone for oxidation stress caused by alcohol.

### [Cell Toxicity]

Firstly, the cell toxicity of lucidone and alcohol is tested. As for test of the cell toxicity of lucidone, HepG2 cells cultivated per the aforementioned cell cultivation methods are classified into 5 groups, and 0, 1, 5, 10 or 20 μg/ml lucidone is separately added into the culture solution for 24 h cultivation; as for test of the cell toxicity of alcohol, HepG2 cells cultivated per the aforementioned cell cultivation methods are classified into 6 groups, and 0, 10, 50, 100, 200 or 500 mM alcohol is separately added into the culture solution for 24 h cultivation. The aforementioned groups without any lucidone and alcohol are control groups of the preferred embodiment (i.e: 0 μg/ml lucidone and 0 mM alcohol). Then, MTT test is carried out to measure the cell viability (refer to preferred embodiment 1 [MTT test]).

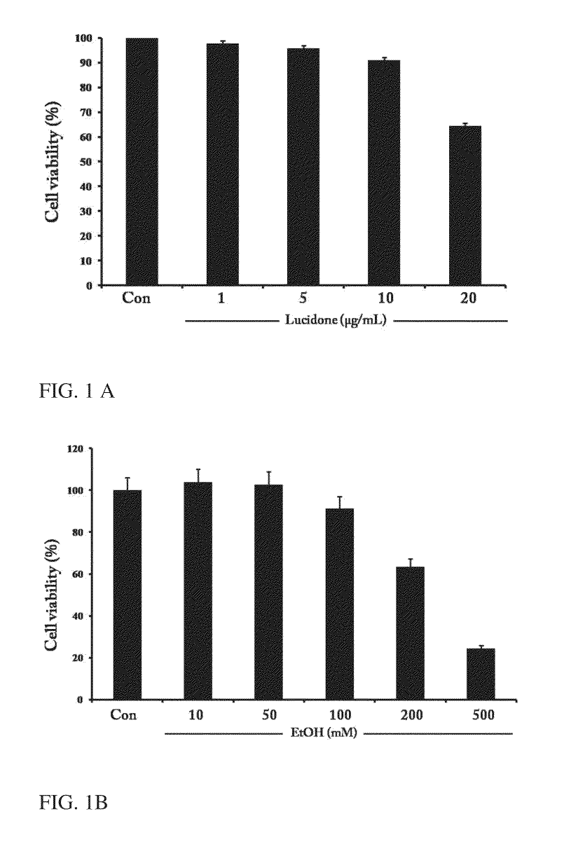

FIG. 1A depicts the test result of lucidone's cell toxicity, showing that 20 μg/ml experiment group has reduced the cell viability about 40% as compared with the control group, but there is not cell toxicity for lucidone of concentration less than 10 μg/ml. FIG. 1B shows that the experiment group begins to generate cell toxicity when alcohol over 100 mM is added.

### [Experimental Design of HepG2 Cell Model]

The aforementioned HepG2 cells cultivated normally are grouped, and then separately cultivated 1 h together with 0, 1, 5, 10 μg/ml lucidone (0 μg/ml, negative control group) or 10 μg/ml curcumin (curcumin, a known antioxidation compound, taken as a positive control group), and then alcohol (100 mM) is added for 24 h cultivation to stimulate oxidation stress. The experiment groups and control groups are configured in Table 1:

TABLE 1
Experimental design of HepG2 cell model

| | | | Alcohol |
|---|---|---|---|
| Category | Lucidone | Curcumin | (EtOH) |
| Blank | − | − | − |
| control | | | |
| group | | | |
| Negative | − | − | +(100 mM) |
| control | | | |
| group | | | |
| Experiment | +(1 μg/ml) | − | +(100 mM) |
| group 1 | | | |
| Experiment | +(5 μg/ml) | − | +(100 mM) |
| group 2 | | | |
| Experiment | +(10 μg/ml) | − | +(100 mM) |
| group 3 | | | |
| Positive | − | +(10 μg/ml) | +(100 mM) |
| control | | | |
| group | | | |

### [Content of ALT, AST and Tumor Necrosis Factor-α in the Cultivated Culture Solution]

The content of ALT, AST and tumor necrosis factor-α (TNF-α) in the tested culture solution of HepG2 cells is measured (refer to preferred embodiment 1 [test of ALT and AST concentration] and [measurement of tumor necrosis factor-α ]).

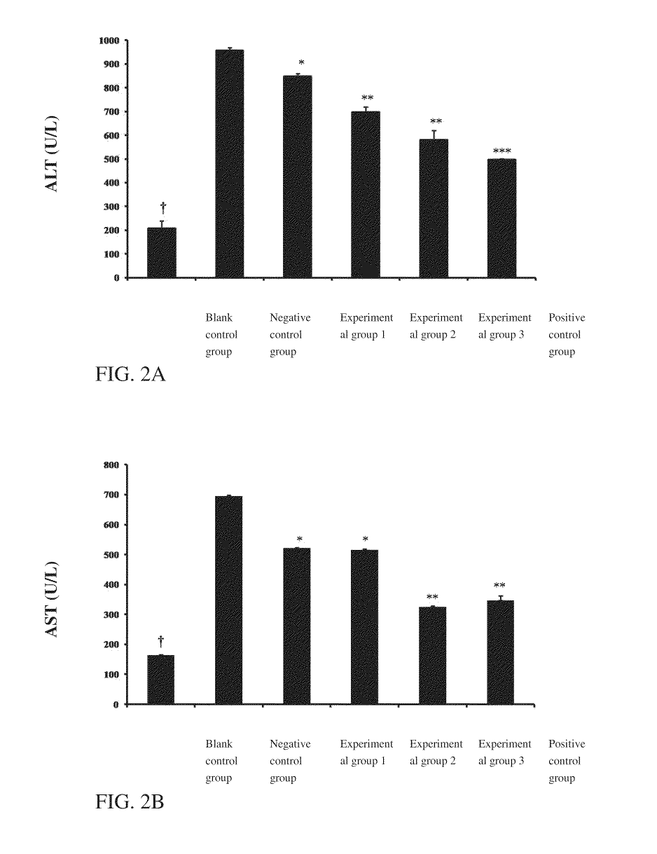

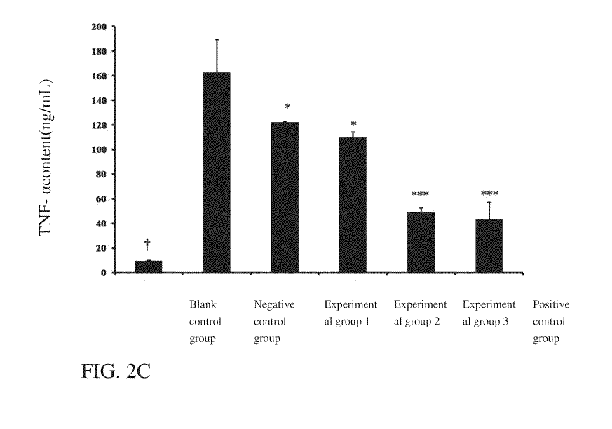

FIGS. 2A-C depict separately the varying content of ALT, AST and TNF-α (of which, con is marked as blank control, and the cells herein are not subject to processing of lucidone, curcumin or alcohol, refer to aforementioned experimental design of HepG2 cell model). As shown in FIGS. 2A-C, the content of ALT, AST and TNF-α in the cell culture solution of negative control group increase obviously, showing that alcohol has aroused inflammatory reaction of HepG2 cells and caused the damage of cells. With a dose-dependent pattern, lucidone has reduced the phenomenon of growing content of ALT, AST and TNF-αcaused by alcohol, with the effect equivalent to the known natural antioxdiation curcumin.

### [Content of Reactive Oxygen Species and Nitric Oxide in the Cultivated Culture Solution]

The content of reactive oxygen species and nitric oxide in the tested culture solution of HepG2 cells is measured (refer to preferred embodiment 1 [Test of accumulation of reactive oxygen species in cells] and [Griess nitrite test]).

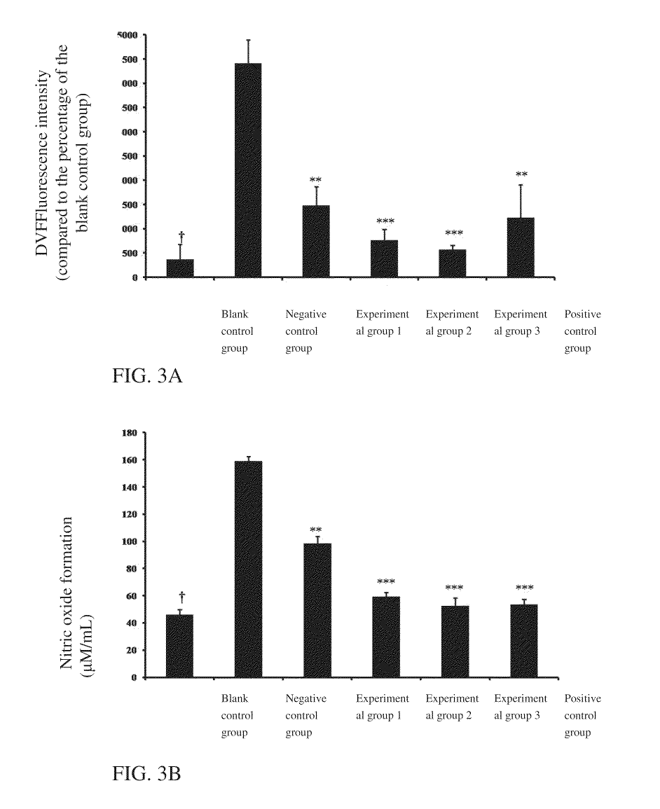

FIG. 3A depicts the content of reactive oxygen species of HepG2 cells in various groups, of which the fluorescence strength of DCF-DA indicates the content of reactive oxygen species. FIG. 3B depicts the generation of nitric oxide of HepG2 cells in various groups, of which nitrite generation indicates the generation of nitric oxide.

FIGS. 3A and B show separately that, the content of reactive oxygen species and nitric oxide in the cell culture solution of negative control group increases significantly, indicating that alcohol arouses oxidation stress of HepG2 cells. As compared with aforementioned experimental results, the growing content of reactive oxygen species and nitric oxide may account for the damage of cells and release of AST and ALT. In addition, with a dose-dependent pattern, lucidone has reduced the generation of reactive oxygen species and nitric oxide caused by alcohol, with the effect equivalent to the known natural antioxdiation curcumin.

### [GSH Consumption and Lipid Peroxidation in Cultivated Culture Solution]

The content of reactive oxygen species and nitric oxide in the tested culture solution of HepG2 cells is measured (refer to preferred embodiment 1 [experimental design of HepG2 cells], [test of lipid peroxidation degree] and [test of glutathione content]).

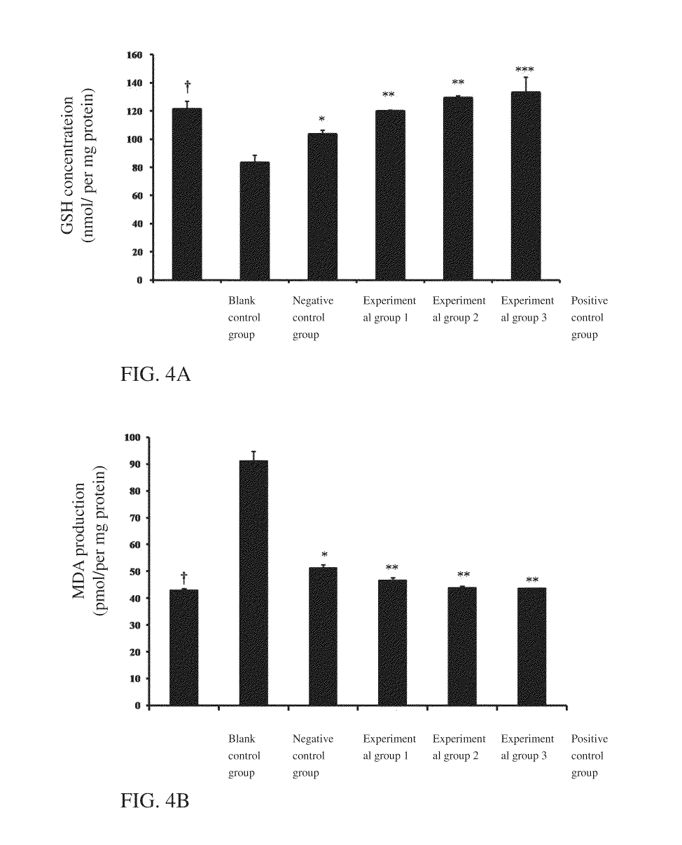

Referring to FIG. 4A, alcohol will lead to the decline of GSH concentration in the cells, indicating that HepG2 cells consume GSH in response to oxidation stress caused by alcohol. This can be avoided by cultivation with lucidone or curcumin, implying the effect of lucidone and curcumin against oxidation stress caused by alcohol.

Referring to FIG. 4B that, alcohol will lead to numerous lipid peroxidation in the cells, but lucidone and curcumin can reduce greatly lipid peroxidation.

[Expression of mRNA and Protein of HO-1 in HepG2 Cells]

Heme oxygenase is a stress-responsive enzyme commonly seen in mammal tissue, which is used for lysing the heme into biliverdin, free iron ions (Fe2+) and carbon monoxide. The expression of heme oxygenase is caused by several stimulants, such as: heme, heavy metal, cytokine, chemical carcinogen and alcohol.

The expression of mRNA and protein of HO-1 in cytosolic extracts of HepG2 cells is measured (refer to relevant experimental methods in preferred embodiment 1).

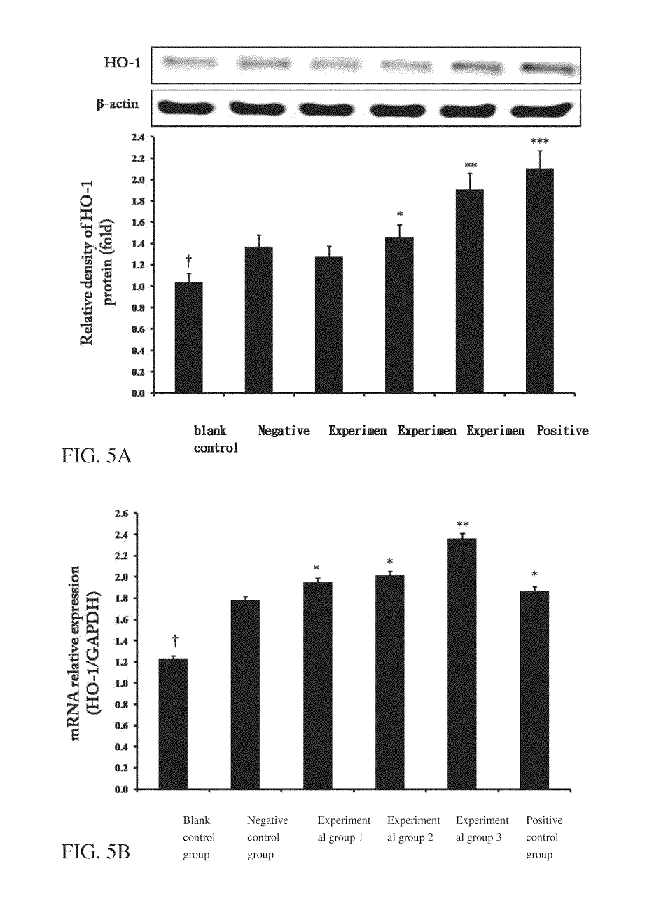

FIGS. 5A and B show that, alcohol will increase the expression of HO-1 protein (FIG. 5A) and mRNA (FIG. 5B) in HepG2 cells; as shown in FIG. 5A, based on the HO-1 protein of blank control group, the relevant strength of HO-1 expression of experiment group, positive and negative control groups can be measured to draw a bar chart below FIG. 5A; said expression refers to the color strength of the result from Western blotting calculated by typical software in the field; FIG. 5B shows that relative HO-1 expression of experiment group, positive and negative control groups is calculated and then drawn, based on mRNA expression of HO-1 in the blank control group.

This result corresponds to the aforementioned test result of cell oxidation stress caused by alcohol. Lucidone and curcumin will cause substantial increase of the expression of HO-1 protein (FIG. 5A) and mRNA (FIG. 5B) in the cells, implying that lucidone can improve the resisting capability of cells against oxidation stress by increasing overall expression of HO-1, like curcumin.

[Translocation of Nrf-2 for HepG2 Cells and its Bonding Activity with ARE]

Transcription factor NF-E2 related factor-2 plays a pivotal role in the expression of ARE (antioxidant response element)-driven antioxidant gene. Transcription factor Nrf-2 is often combined with its Keap-1 to form inactive compound and segregated into cytoplasm. Once stimulated by oxidation stress, the transcription factor Nrf-2 will be segregated from its Keap-1 and activated; then, the activated transcription factor Nrf-2 will be translocated into the nucleus, and then combined with ARE on the promoter of target gene to facilitate the performance of the target gene:

HO-1 is one of ARE-driven antioxidant genes. Based on the test results that, lucidone can improve the expression of mRNA and protein of HO-1. In this experiment, the content of Nrf-2 in cytoplasm and nuclear extracts of tested HepG2 cells, as well as the bonding activity of Nrf-2 and ARE is measured (refer to relevant experimental methods in preferred embodiment 1).

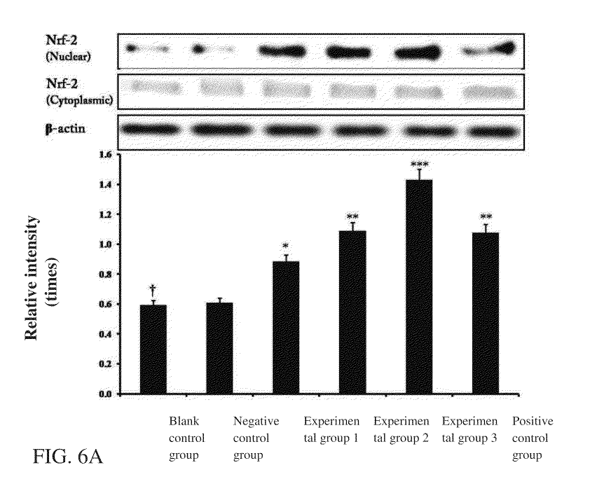

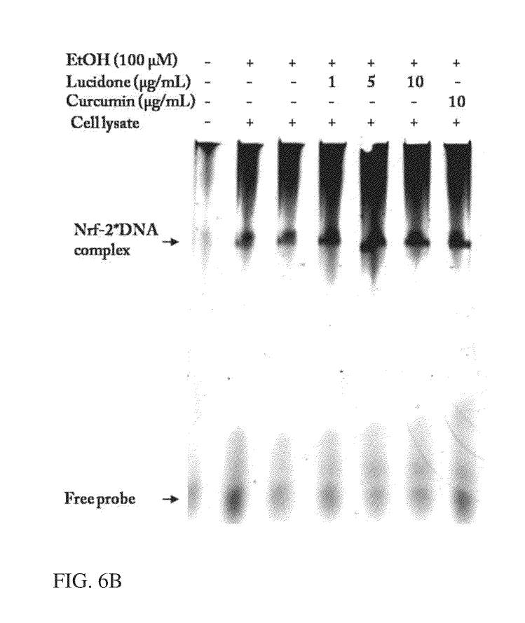

FIG. 6A shows that Western blotting is used to measure the content of Nrf-2 in cytoplasm and nucleus, of which the deep color represents higher content of Nrf-2; based on Nrf-2 content in nucleus of blank control group, the relevant quantity of Nrf-2 content in nucleus of experiment group, positive and negative control groups can be measured to draw a bar chart below FIG. 6A. Said content refers to the color strength of the result from Western blotting calculated by typical software in the field. The result indicates that, alcohol has not resulted in significant Nrf-2 translocation, but lucidone will facilitate Nrf-2 translocation, implying that lucidone will drive the activation of Nrf-2 and improve antioxidation capability of cells.

FIG. 6B shows that Electrophoretic mobility shift assay (EMSA) is used to measure the bonding activity of Nrf-2 and ARE. The results indicate that, lucidone can improve significantly the bonding activity of Nrf-2 and ARE with a dose-dependent mode, which is consistent with the test results of increasing Nrf-2 translocation.

### PREFERRED EMBODIMENT 3

### Discussion of the Effects of Lucidone Against Alcoholic Oxidation Stress and Hepatitis with Mouse Model

### [Experimental Design]

The aforementioned mouse are grouped and then applied with lucidone for 7 d at a dose of 100 mg/Kg/day by means of intra-peritoneal injection, and at the 7^{th} day, applied with lucidone and then applied with 80% alcohol orally or by intra-peritoneal injection at a dose of 1 ml/Kg, one time every 12 h with 3 doses; of which, the mouse of the control group is fed with 1% DMSO dissolved in saline solution.

### [Content of ALT, AST, Nitric Oxide and TNF-α in the Serum of Tested Mouse]

Take out the serum of mouse suffered by hepatitis arising from alcohol as per aforementioned experimental design, and determine the content of ALT, AST, nitric oxide and TNF-α by the experimental methods in preferred embodiment 1. The experimental results are listed in Table 2:

TABLE 2
Determination of content of ALT, AST,
nitric oxide and TNF-α in mouse model

| | | | |
|---|---|---|---|
| | | | Alcohol and |
| | | Only alcohol | lucidone |
| Category | Control group | applied | applied |

Orally taking alcohol

| | | | |
|---|---|---|---|
| AST (U/L) | 31.9 ± 18.4† | 1542.8 ± 163.8 | 508.0 ± 31.7 |
| ALT (U/L) | 61.2 ± 65.6† | 3053.4 ± 464.5 | 875.4 ± 57.2 |
| Nitric | 138.6 ± 0.54† | 1312.1 ± 0.76 | 370.6 ± 0.53 |
| oxide(nM/mL) | | | |
| TNF-α (ng/mL) | 1.64 ± 0.38† | 68.9 ± 0.72 | 10.0 ± 0.28 |

Intra-peritoneal injection alcohol

| | | | |
|---|---|---|---|
| AST (U/L) | 83.8 ± 17.4† | 314.8 ± 181.0 | 139.6 ± 12.2* |
| ALT (U/L) | 69.25 ± 26.0† | 332.3 ± 41.4 | 103.0 ± 65.6* |
| nitric | 79.6 ± 0.50† | 160.7 ± 0.59 | 102.3 ± 0.52* |
| oxide(nM/mL) | | | |
| TNF-α (ng/mL) | 3.79 ± 0.60† | 15.6 ± 0.77 | 3.91 ± 0.28* |

The value in the table is represented by ± standard deviation, and every group is available with 5 experimental mouse in the experiment.
†represents the data by comparing control groups with those only applied with alcohol, with the differential P value ≦ 0.01
*represents the data of by comparing the groups applied only with alcohol and those applied with alcohol and lucidone, with the differential P value ≦ 0.01

According to the test data of mouse not applied with lucidone as listed in Table 2, alcohol will cause hepatotoxicity in the mouse, which is attributable to the growing content of ALT, AST, nitric oxide and TNF-α in the serum. In the serum of mouse applied with lucidone in advance, the content of ALT, AST, nitric oxide and TNT-a varies little as compared with the control group, showing the effect of lucidone in preventing mouse hepatitis caused by alcohol. This result corresponds to the experimental results of HepG2 cells in aforementioned preferred embodiment.

### [GSH Content and Lipid Peroxidation Degree of Tested Mouse]

Take out the serum and liver tissue of mouse suffered by hepatitis arising from alcohol as per aforementioned experimental design, and measure GSH content and lipid peroxidation degree as per the experimental methods in preferred embodiment 1. The results are listed in Table 3:

TABLE 3
Determination of content of GSH and MDA
in serum and tissue in mouse model

| | | | |
|---|---|---|---|
| | | | Alcohol and |
| | | Only alcohol | lucidone |
| Category | Control group | applied | applied |

Orally taking alcohol

| | | | |
|---|---|---|---|
| GSH in serum | 34.5 ± 7.2† | 23.6 ± 15.8 | 30.5 ± 1.0* |
| (nmol/mg of | | | |
| protein) | | | |
| MDA in serum | 12.6 ± 0.36† | 52.8 ± 0.60 | 19.5 ± 0.41* |
| (nmol/mg of | | | |
| protein) | | | |
| GSH in tissue | 138.6 ± 0.54† | 165 ± 4.5 | 261.2 ± 11.1* |
| (nmol/mg of | | | |
| protein) | | | |
| MDA in tissue | 1.64 ± 0.38† | 142.3 ± 0.75 | 69.2 ± 0.67* |
| (nmol/mg of | | | |
| protein) | | | |

Intra-peritoneal injection alcohol

| | | | |
|---|---|---|---|
| GSH in serum | 41.1 ± 2.9† | 18.4 ± 4.3 | 42.9 ± 1.8* |
| (nmol/mg of | | | |
| protein) | | | |
| MDA in serum | 15.6 ± 0.40† | 41.2 ± 0.77 | 16.2 ± 0.77* |
| (nmol/mg of | | | |
| protein) | | | |
| GSH in tissue | 217.3 ± 83.2† | 51 ± 5.7 | 173.9 ± 58.5* |
| (nmol/mg of | | | |
| protein) | | | |
| MDA in tissue | 15.6 ± 0.75† | 45.1 ± 0.58 | 20.9 ± 0.71* |
| (nmol/mg of | | | |
| protein) | | | |

The value in the table is represented by ± standard deviation, and every group is available with 5 experimental mouse in the experiment.
†represents the data by comparing control groups with those only applied with alcohol, with the differential P value ≦ 0.01
*represents the data of by comparing the groups applied only with alcohol and those applied with alcohol and lucidone, with the differential P value ≦ 0.01

The experimental results in Table 3 show that, GSH content of mouse applied with alcohol has a declining trend, as this case may be prevent if lucidone is applied in advance. In addition, MDA concentration of mouse serum applied with alcohol will rise, implying the increase of lipid peroxidation degree; but lipid peroxidation degree could be reduced by applying lucidone in advance. Hence, the mouse applied with lucidone has stronger capability of resisting oxidation stress caused by alcohol as compared with that not applied with lucidone.

1. A pharmaceutical composition for hepatitis treatment, which comprises efficient amount of lucidone as an active ingredient;
and pharmaceutical acceptable carrier.

2. The composition defined in claim 1, wherein said hepatitis refers to alcoholic hepatitis.

3. The pharmaceutical composition as claimed in claim 1, wherein it comprises 1˜2 wt % lucidone, and 98˜99 wt % pharmaceutical acceptable carrier.

4. The composition as claimed in claim 1, wherein said carrier is starch, lactose, cane sugar, MCC or CMC.

5. The composition as claimed in claim 1, wherein said additive is amino acid, vitamin or mineral substance.

6. The composition as claimed in claim 1, wherein it refers to troche, capsule, skin patch, suspending agent, powder, nasal inhaler, spray or injection.

7. A method of reducing oxidation stress caused by alcohol, which enables contact of the sample with either of said pharmaceutical composition defined in claims 1˜6.

8. The method of reducing oxidation stress caused by alcohol as claimed in claim 7, wherein said samples refer to mammals.

9. The method of reducing oxidation stress caused by alcohol as claimed in claim 7, wherein said mammals refer to the human beings.
